SEQUENCE LISTING (3 sequences)

SEQ ID NO: 1 - DNA, 19 bp, Artificial Sequence
  primer sequences
cataacgagcggaagaacg

SEQ ID NO: 2 - DNA, 64 bp, Artificial Sequence
  primer sequences
gatccccgggaagttctggtgtcatattcaagagatatgacaccagaacttccctttttggaaa

SEQ ID NO: 3 - DNA, 64 bp, Artificial Sequence
  primer sequences
agcttttccaaaaagggaagttctggtgtcatatctcttgaatatgacaccagaacttcccggg

(19) United States
(12) Patent Application Publication
(10) Pub. No.: US 2012/0202883 A1
(43) Pub. Date: Aug. 9, 2012

(54) EFFECTS OF IDH1 AND IDH2 MUTATIONS ON THE CELLULAR METABOLOME

(75) Inventors: Yan Hai, Chapel Hill, NC (US); Darell Bigner, Mebane, NC (US); Yiping He, Chapel Hill, NC (US); Genglin Jin, Durham, NC (US); Zachary Reitman, Durham, NC (US)

(73) Assignee: DUKE UNIVERSITY, Durham, NC (US)

(21) Appl. No.: 13/355,129

(22) Filed: Jan. 20, 2012

Related U.S. Application Data
(60) Provisional application No. 61/434,716, filed on Jan. 20, 2011.

(51) Int. Cl. A61K 31/191; A61K 31/197; A61P 35/02; C12Q 1/68; A61P 35/00

(52) U.S. Cl. 514/578; 435/6.11

(57) ABSTRACT

Point mutations of the NADP⁺-dependent isocitrate dehydrogenases (IDH1 and IDH2) occur early in the pathogenesis of gliomas. When mutated, IDH1 and IDH2 gain the ability to produce the metabolite (R)-2-hydroxyglutarate (2HG), but the downstream effects of mutant IDH1 and IDH2 proteins or of 2HG on cellular metabolism are unknown. Here, we profiled >200 metabolites in human oligodendroglioma cell line (HOG) cells to determine the effects of expression of IDH1 and IDH2 mutants. Levels of amino acids, glutathione metabolites, choline derivatives, and tricarboxylic acid (TCA) cycle intermediates were altered in both mutant IDH1- and IDH2-expressing cells. These changes were similar to those identified after treatment of the cells with 2HG. Remarkably, N-acetyl-aspartyl-glutamate (NAAG), a common dipeptide in brain, was 50-fold reduced in cells expressing IDH1 mutants and 8.3-fold reduced in cells expressing IDH2 mutants. NAAG was also significantly lower in human glioma tissues containing IDH mutations than in gliomas without such mutations.

This invention was made using funds from the U.S. government. The U.S government retains certain rights in the invention under the terms of grants NIH 5P30-CA-014236-36, and NCI Grant R01-CA-140316.

### TECHNICAL FIELD OF THE INVENTION

This invention is related to the area of cancer. In particular, it relates to metabolic changes in cancer cells.

### BACKGROUND OF THE INVENTION

Differences in cellular metabolism between cancer and normal cells have long been noted by cancer researchers (1). Genetic alterations that occur in cancer, such as mutations and copy number changes that alter K-Ras and c-Myc, are thought to be responsible for at least some of these metabolic differences (2, 3). Thus, the genetic alterations that drive cancer pathogenesis may do so in part by altering cellular metabolism, which could aberrantly signal cells to proliferate and provide molecular building blocks for cellular replication (4). This has generated enthusiasm for the idea that that drug targets for the specific killing of cancer cells can be identified by studying the metabolic differences between normal and cancer cells.

Gliomas are tumors of the central nervous system that respond poorly to therapy and are associated with a heterogeneous collection of genetic alterations (5, 6), including mutations in IDH1 and IDH2 (7, 8). IDH1 and IDH2 are the cytoplasmic and mitochondrial NADP⁺-dependent isocitrate dehydrogenases, respectively, and are homologs. IDH3, which is unrelated to IDH1 and IDH2, is the NAD⁺-dependent isocitrate dehydrogenase and has not been found to be mutated in cancer (FIG. S1A). These enzymes convert isocitrate to α-ketoglutarate (FIG. S1B). IDH1 catalyzes this reaction in the cytosol and peroxisome to mediate a variety of cellular housekeeping functions, while IDH2 and IDH3 catalyze a step in the TCA cycle (reviewed in 9). IDH1 R132 mutations occur frequently (50-93%) in astrocytomas and oligodendrogliomas, as well as in secondary glioblastomas and may be the initiating lesion in these glioma subtypes (7, 8). Mutations in the analogous IDH2 R172 codon also occur at a lower rate (3-5%) in these cancers (8). Mutations in IDH1 and IDH2 have also been observed in 22% of acute myelogenous leukemias (10). In gliomas, R132H is the most common IDH1 mutation, and R172K is the most common IDH2 mutation (8). IDH1 and IDH2 mutations are mutually exclusive and alter only one allele, apparently in a dominant fashion (8, 11). These observations suggest that IDH1 and IDH2 are proto-oncogenes that are activated by mutation of R132 and R172, respectively. Mutation of these codons abolishes the normal ability of IDH1 and IDH2 to convert isocitrate to α-ketoglutarate (8). Also, the mutated IDH1-R132H enzyme can dominant-negatively inhibit IDH1-WT isocitrate dehydrogenase activity in vitro (12). This has led to the suggestion that an oncogenic function for the IDH mutations is to dominant negatively inhibit IDH1 enzymatic activity. A separate line of research revealed that IDH1 R132 and IDH2 R172 mutants gain the neomorphic ability to convert α-ketoglutarate to 2HG (FIG. S1B), and that 2HG is highly elevated in IDH-mutated cancer tissues (10, 13, 14). There is a continuing need in the art to.

### SUMMARY OF THE INVENTION

One aspect of the invention is a method of characterizing a brain cell sample or blood cell sample. The sample is tested for amount of N-acetyl-aspartyl-glutamate (NAAG) or N-acetyl-aspartate (NAA). The amount of NAAG or NAA in the sample is compared to the amount in corresponding normal cells of the same individual or to similar cells of a control individual that has an IDH1^{+/+}/IDH2^{+/+}genotype. A sample with a reduced amount of NAAG or NAA likely has an IDH1 R132 or IDH2 R172 mutation.

Another aspect of the invention is a method of characterizing a brain cell sample, a blood cell sample, a cerebrospinal fluid sample, or blood plasma sample. The sample is tested for amount of a metabolite selected from the group consisting of kynurenine, phosphocholine, glycerophosphocholine, 4-methyl-2-oxopentanoate, 3-methyl-2-oxovalerate, and 3-methyl-2-oxobutryate. The amount of the metabolite in the sample is compared to the amount in a control sample from an individual (or individuals) that has an IDH1^{+/+}/IDH2+^{/+} genotype. A sample with an increased amount of kynurenine, phosphocholine, or glycerophosphocholine, or a reduced amount of 4-methyl-2-oxopentanoate, 3-methyl-2-oxovalerate, and 3-methyl-2-oxobutryate, likely has a IDH1 R132 or IDH2 R172 mutation.

An additional aspect of the invention is a method of treating a cancer in an individual. An agent is administered to the individual. The agent is selected from the group consisting of 2-hydroxyglutarate, N-acetyl-aspartate, or N-acetyl-aspartyl-glutamate. The treatment may reduce the amount of the cancer, the growth rate of the cancer, the anatomical spread of the cancer.

These and other embodiments which will be apparent to those of skill in the art upon reading the specification provide the art with tools for diagnosing, characterizing, and treating cancers, particularly those having IDH1 or IDH2 mutations.

### BRIEF DESCRIPTION OF THE DRAWINGS

FIG. 1A-1C. Metabolite profile of a glioma cell line expressing IDH1-R132H or IDH2-R172K. FIG. 1A, Heat map showing 314 biochemicals in lysates from six replicates each of HOG cells expressing IDH1-WT, IDH1-R132H, IDH2-WT, IDH2-R172K, or vector alone, arranged by unsupervised hierarchical clustering. The level of each biochemical in each sample is represented as the number of standard deviations above or below the mean level of that biochemical (z score). FIG. 1B, Venn diagrams indicating the number of biochemicals with mean levels that are significantly (p<0.05) higher or lower in cells expressing each transgene compared to the vector. FIG. 1C, PCA of metabolite profile dataset. The percentage of variance in the dataset reflected by the first 6 PCs is shown in the histogram, and PC1 and PC2 for each sample are plotted.

FIG. 2A-2C. Metabolite profile of a glioma cell line expressing IDH1-R132H, treated with 2HG, or with knocked-down IDH1. FIG. 2A, Heat map showing z scores for 202 unique biochemicals of known identity in HOG cell lysates, arranged by unsupervised hierarchical clustering. Six replicates each of cells stably expressing IDH1-R132H, IDH1 shRNA, or scramble shRNA, and cells treated with media containing 0 (vector), 7.5 mM, or 30 mM 2HG for 72 hours prior to analysis are shown. FIG. 2B, Venn diagrams indicating the number of biochemicals with mean levels that are significantly (p<0.05) higher or lower in each group of cells compared to vector, and the number of these changes shared between cells in the indicated groups. FIG. 2C, PCA of this metabolite profile dataset. The percentage of variance in the dataset reflected by the first 6 PCs is shown in a histogram and PC1 and PC2 for each sample are plotted.

FIG. 3. Metabolites altered by 2-fold or more by IDH1-R132H expression. Biochemicals that were on average >2-fold higher or lower in HOG IDH1-R132H cells relative to vector cells are displayed. The fold-change of these biochemicals in cells expressing IDH1-WT, IDH2-R172K, IDH2-WT, treated with 2HG, or expressing IDH1 shRNA is also shown. All >2-fold changes shown here were significant (p<0.05). Note that this scale colors only those findings with >2-fold change. Detailed information on these changes can be found in Tables S1 and S4.

FIG. 4A-4E. Alterations in metabolic pathways observed in cells expressing IDH1-R132H, expressing IDH2-R172K, or treated with 2HG. The fold difference in metabolite in each experiment relative to vector is indicated by the color of each box (IDH1-R132H, left boxes; IDH2-R172K, middle boxes; or 30 mM 2HG, right boxes). FIG. 4A, Amino acids and N-acetylated amino acids. FIG. 4B, BCAAs and catabolites. FIG. 4C, Glutathione and metabolites involved in its regeneration. FIG. 4D, Choline, GPC, and intermediates. FIG. 4E, TCA and shuttling of citrate, isocitrate, and a-ketoglutarate to the cytosol. Dashed lines indicate exchange of a metabolite between the mitochondria and cytosol. γ-glu-aa, γ-glutamyl amino acids; aa, amino acids; γ-glu-cys, γ-glutamyl-cysteine; cysH-gly, cysteinylglycine.

FIG. 5A-5C. NAA and NAAG in cell lines and tumors containing IDH1-R132H determined by targeted LC-MS. FIG. 5A, NAA and NAAG levels in HOG cells expressing vector, IDH1-WT, or IDH1-R132H incubated in mock, 10004 NAA, 1004 NAAG media for 48 hours. FIG. 5B, NAA and NAAG in media incubated for 48 hours above HOG cells expressing IDH1-R132H, IDH1-WT, or a vector control. FIG. 5C, NAA and NAAG levels in human glioma tissues with IDH1-R132H mutation (n=17) and without IDH mutations (WT, n=9). *p<0.05, **p<0.005.

FIG. 6A-6F (S1). Overview, validation, and additional data for metabolomic analysis of HOG cells expressing IDH1 and IDH2 transgenes. FIG. 6A, Normal cellular localization of the isocitrate dehydrogenases IDH1, IDH2, and IDH3. FIG. 6B, Enzymatic function of WT IDH1 and IDH2, and of cancer-derived IDH1 and IDH2 mutants such as IDH1-R132H and IDH2-R172K. FIG. 6C, 5 clones of HOG cells were stably transduced with a lentivirus vector to express different transgenes. The transgenes include IDH1-WT, IDH1-R132H, IDH2-WT, IDH2-R172K, and an empty vector (V) control. Six replicate samples of each group were grown for analysis. FIG. 6D, Anti-V5 and anti-GAPDH immunoblot of HOG clones described in C. FIG. 6E, Plots of PC3-PC6 from PCA analysis of replicate samples from C. FIG. 6F, PC1 loading values for 314 biochemicals, arranged in order of increasing loading value. The 5 biochemicals with the highest and lowest loading values are listed.

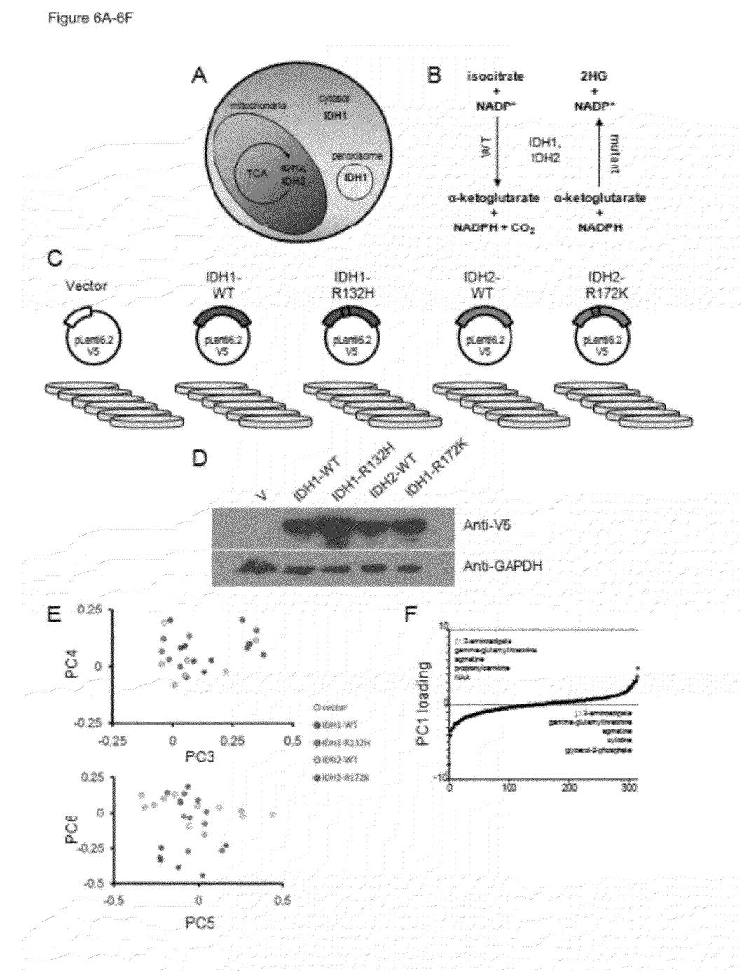

FIG. 7 (S2). Summary of technical and statistical methods used in metabolomic profiling experiments in this study. Samples were generated from a human glioma cell line as described in the text and analyzed by LC-MS (+/−ESI) and GC-MS (-EI). They were then subjected to multivariate and univariate statistical analyses to identify global and specific metabolite differences between different transgene expression and experimental treatment groups as shown.

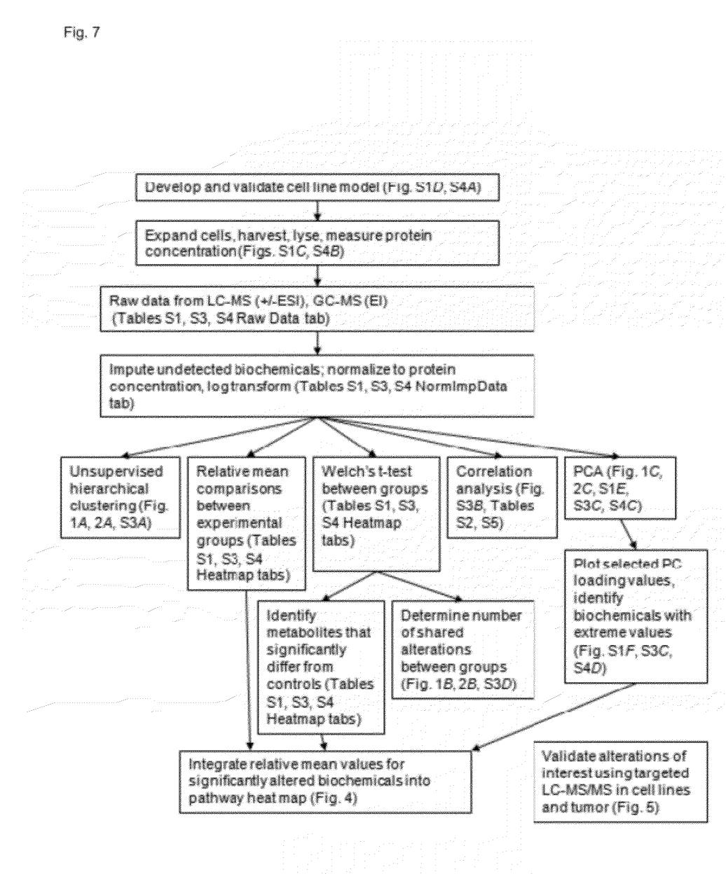

FIG. 8A-8D (S3). Abundance of metabolites in media incubated above cells expressing IDH1-R132H. FIG. 8A, Heat map showing levels of 111 biochemicals in media incubated above HOG cells expressing IDH1-WT, IDH1-R132H, or an empty vector. Samples are arranged according to a dendrogram generated using unsupervised hierarchical clustering. FIG. 8B, Table showing Pearson product-moment correlation coefficients for between the mean relative abundances of biochemicals in each group of media samples. FIG. 8C, PCA of metabolite profile dataset. The percentage of variance in the dataset reflected by the first 6 PCs is shown in the histogram, and PC1-PC6 for each sample are plotted. FIG. 8D, Six metabolites were identified that had a significant (p<0.05) difference in mean level between the IDH1-R132H group and both the IDH1-WT and vector groups. The relative level of these metabolites in the IDH1-R132H group compared to the vector control and to fresh media are shown. These metabolites had similar levels in the vector and IDH1-WT groups (Table S3), so the IDH1-R132H:IDH1-WT comparison is not displayed.

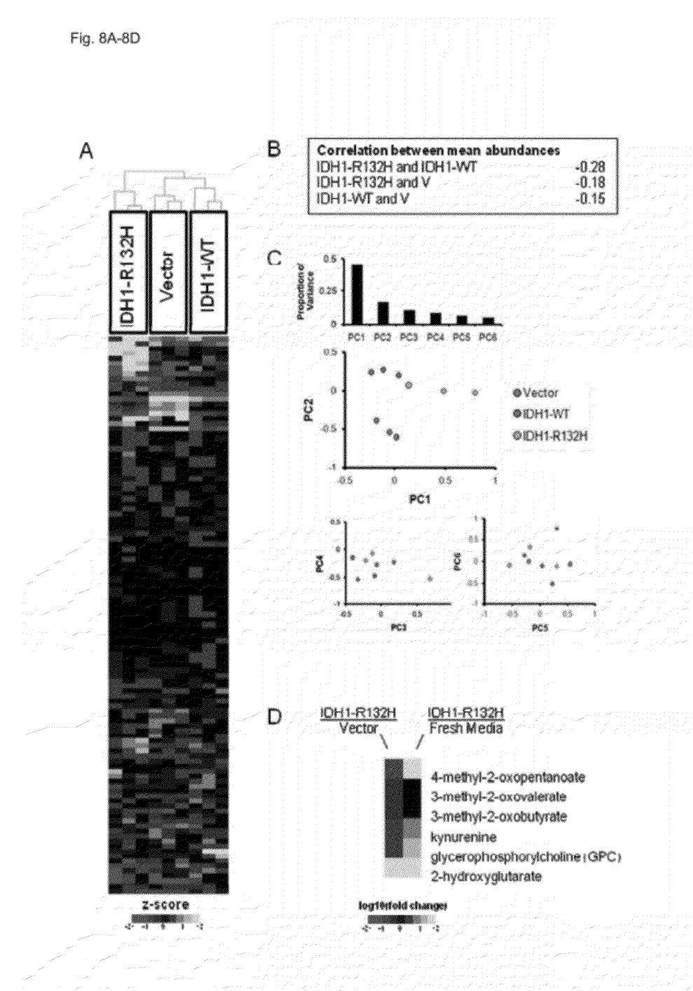

FIG. 9A-9D (S4). Overview, validation, and additional data for metabolomic analysis of HOG cells expressing IDH1-R132H, with IDH1 knockdown, or with 2HG treatment. FIG. 9A, Anti-IDH1 immunoblot of HOG cells stably expressing scrambled shRNA or shRNA targeted against IDH1. IDH1 and IDH2 both have NADP⁺-dependent isocitrate dehydrogenase activity. HOG cells with IDH1 shRNA have 50% lower NADP⁺-dependent isocitrate dehydrogenase activity at 2 mM isocitrate. This is consistent with near-total IDH1 knockdown, with the remaining 50% of activity possibly derived from endogenous IDH2. FIG. 9B, The following types of cells were analyzed: vector, without treatment or treated with 7.5 mM or 30 mM 2HG, cells expressing IDH1-R132H, cells expressing shRNA targeted to IDH1 or scrambled control shRNA. FIG. 9C, Plots of PC5-PC8 from PCA analysis of replicate samples of 6 HOG cell treatments in Experiment 2. FIG. 9D, PC1 loading values for 202 biochemicals, arranged in order of increasing loading value. The 5 biochemicals with the highest and lowest loading values for PC1 are listed.

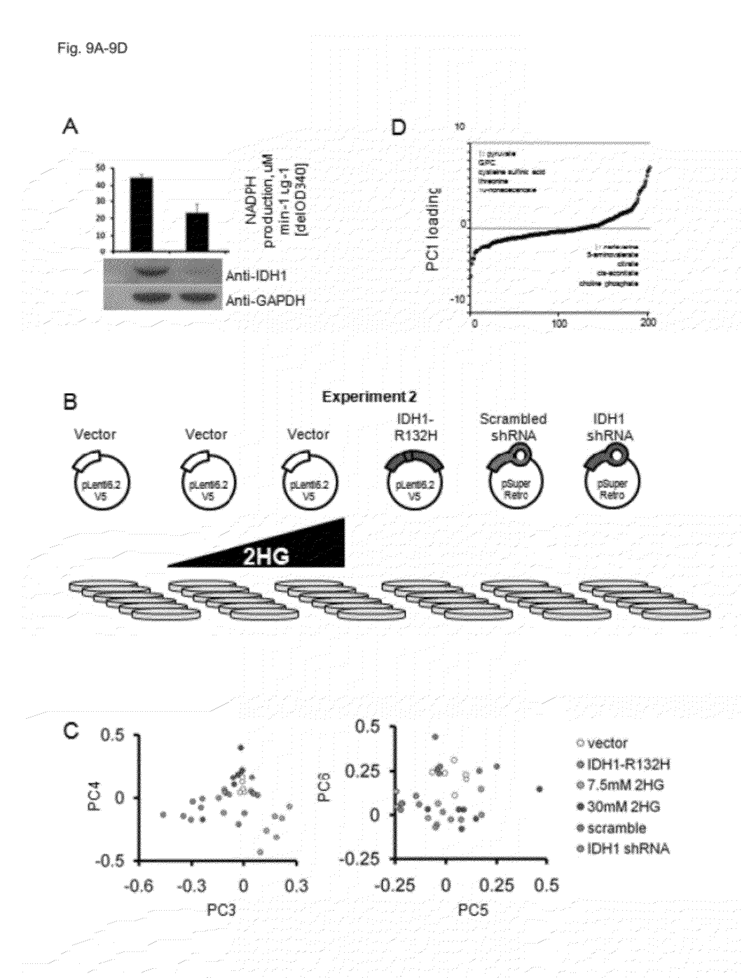

FIG. 10. NAA and NAAG abundance in human glioma tissue. Tumor type, IDH mutation status, NAA and NAAG abundance expressed in ng/mg protein are shown for 26 tumor specimens from human glioma patients.

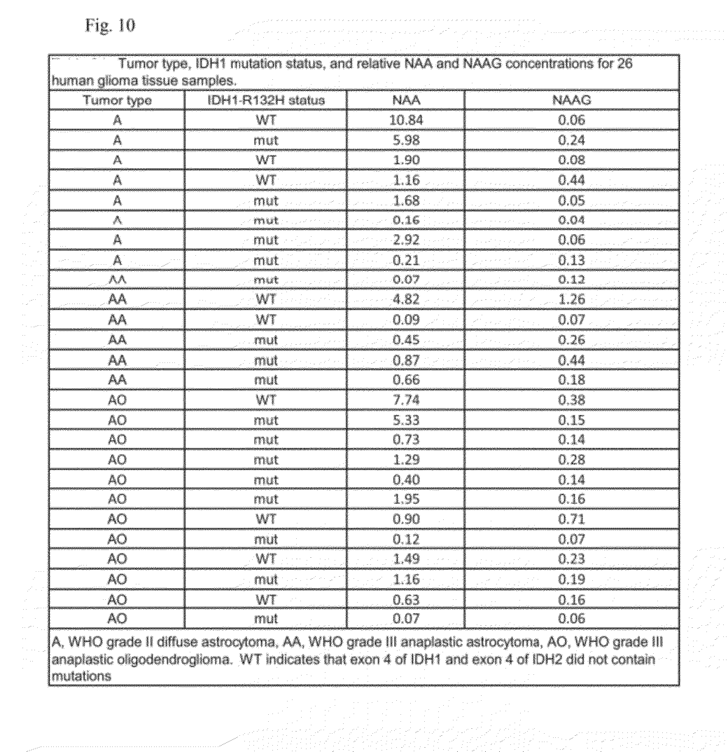

### DETAILED DESCRIPTION OF THE INVENTION

The inventors have developed methods for diagnosing, characterizing, and treating based on metabolic changes that occur in certain cancers. The metabolic changes are characteristic of the cancers and distinguish the cancers from normal cells. Similarly, some of the metabolic changes are reflected in secreted products into body fluids, such as blood, lymph, cerebrospinal fluid, etc. The body fluids can also be tested to detect these metabolic changes. Altering levels of some of the secreted metabolites by applying exogenous agents can be used as a means of treating cancers.

Cancers to which the methods can be applied include those which have IDH1 R132 or IDH2 R172 mutations. Cancers with other IDH mutations may also be susceptible to the methods. Although typically such mutations have been found in brain tumors such as glioblastomas, astrocytoma, oligodendroglial tumor, as well as in acute myelogenous leukemia, other cancers may also be susceptible to the methods, particularly the therapeutic methods. These include, without limitation, fibrosarcoma, paraganglioma, prostate cancer, acute lymphoblastic leukemia, breast cancer, colon cancer, lung cancer, ovarian cancer, kidney cancer, uterine cancer, cervical cancer, testicular cancer, liver cancer, pancreatic cancer, esophageal cancer, bladder cancer, melanoma, gastrointestinal stroma cancer, thyroid cancer.

Samples which can be tested for characterization include brain cells samples and blood cell samples. The samples can be obtained, for example, by digestion of tissue samples and pulverization of cells to lyse them. The cells may be obtained by centrifugation of whole blood to isolate cells. Cell can be lysed by any means known in the art. Cells can be purified prior to lysis, as they may be present in tissues of a mixed nature. Any cell isolation, purification, and lysis methods can be used. Similarly, body fluids can be analyzed for secreted or leaked metabolites. Such body fluids include, without limitation, blood, fractionated blood, such as serum or plama, urine, stool, sputum, tears, saliva, cerebrospinal fluid, lymph, nipple aspirate, breast milk, semen.

Control samples can be from the same individual using a matched sample. For example, if the test sample is from brain tissue that appears to be neoplastic or abnormal, then the control sample can be from a brain tissue that appears to be normal of the same individual. If the sample is from a body fluid, such as blood, then the control sample can be from an individual without disease, an individual without IDH mutations, a pooled sample from normal, healthy individuals, or pooled data from normal individuals. Preferably control and test samples will be similarly prepared so that quantitative comparisons are valid and meaningful.

Increases or decreases in levels of metabolites will vary with the metabolite and with the individual and the disease. In some cases the change may be only about 1.5-fold, and in other cases changes may be about 50-fold, for example. Threshold differences may be set at (at least) 1.5-fold, 2-fold, 4-fold, 5-fold, 7.5-fold, 10-fold, 15-fold, 20-fold, 25-fold, 30-fold, 35-fold, 40-fold, 45-fold, 50-fold, as non-limiting examples.

Levels of metabolites may be tested using any available technologies and techniques. Although particular technologies are described below for measuring metabolites, others can be used as is convenient or beneficial in a particular setting. Analyses may be conducted using liquid chromatography (LC), gas chromatography (GC), mass spectrometry (MS), or combinations of these, for example. A platform can be used that screens for a very large number of metabolites or a targeted platform can be used for those metabolites identified here as relevant. Metabolites which may be tested include but are not limited to N-acetyl-aspartyl-glutamate (NAAG), N-acetyl-aspartate (NAA), kynurenine, phosphocholine, glycerophosphocholine, 4-methyl-2-oxopentanoate, 3-methyl-2-oxovalerate, and 3-methyl-2-oxobutryate.

IDH mutation status can be determined genetically by any technique known in the art. Exon 4 of each of IDH1 and IDH2 can be analyzed to identify changes in codons 132 or 172 respectively. Any type of genetic assay can be used, including nucleotide sequencing, hybridization to probes, primer-specific amplification, single nucleotide extension, etc. Genetic analysis can be used to confirm a metabolic analysis. It can also be used to determine subjects for therapeutic, metabolic treatment.

Characterization of the IDH1/IDH2 status of a sample can be used to provide a diagnosis, to provide a prognosis, and to prescribe an appropriate anti-cancer therapy. The characterization may not be the only factor considered, but may be combined with a physician's clinical judgments and assessments. Other factors may include radiological data, histological data, physical examination findings, other biochemical markers such as genetic, epigenetic, or protein markers, age, gender, etc. Diagnoses, prognoses, and prescriptions of therapy may be formulated in the brain of a human or in a computer. However, such conclusions are communicated by a physical act, such as recording in a chart or medical record, recording on a paper or electronic prescription, orally communicating to a patient, family member, or other member of a medical treatment team. Mutations in IDH1 and IDH2 are positive prognostic indicators, for example, occurring in low grade, diffuse astrocytomas and in secondary glioblastomas. Moreover, the mutations may sensitize the tumors to chemotherapy or radiation therapy.

Administration of a metabolite to an individual with cancer can be accomplished by any means known in the art, including without limitation, intravenous, intramuscular, intratumoral, liposomal, targeted liposomal, liposome and sonar, oral, implantation of pellets or other impregnated solid, intrathecal. The administration may be systemic, targeted, or local. Infusions or injections may be used. The metabolite treatment may be combined with other therapeutic modalities, including but not limited to chemotherapy, surgical removal, stem cell transplantation, radiation therapy, biological therapy including antibodies or T cells. Examples of metabolites which can be used therapeutically are N-acetyl-aspartyl-glutamate (NAAG), 2-hydroxyglutarate, N-acetyl-aspartate, and combinations of these. The metabolites may be racemic mixtures, or the L- or D-forms. As we show below, exogenously administered 2-hydroxyglutarate can induce metabolic changes in cancer cells. Although applicants do not wish to limit themselves to any particular theory or mechanism of operation, such metabolic changes could be toxic to the cancer cells, particularly to those with IDH1 or IDH2 mutations.

IDH1-R132H expression and IDH2-R172K expression induce multiple changes in the cellular metabolome. Exogenous 2HG, or IDH1-WT knockdown, can have similar effects to IDH1-R132H expression. Knockdown of IDH1-WT produced few changes that were also caused by IDH1-R132H expression, indicating that dominant negative inhibition of the functional IDH1 allele by IDH1-R132H may not have a large effect on the metabolome of glioma cells. In contrast to this, 2HG treatment results in a more similar global metabolic changes to IDH1-R132H expressing cells than to controls, and 2HG treatment and IDH expression both associate with similar changes in many metabolites in specific pathways, including free amino acids, BCAAs, and choline phospholipid synthesis.

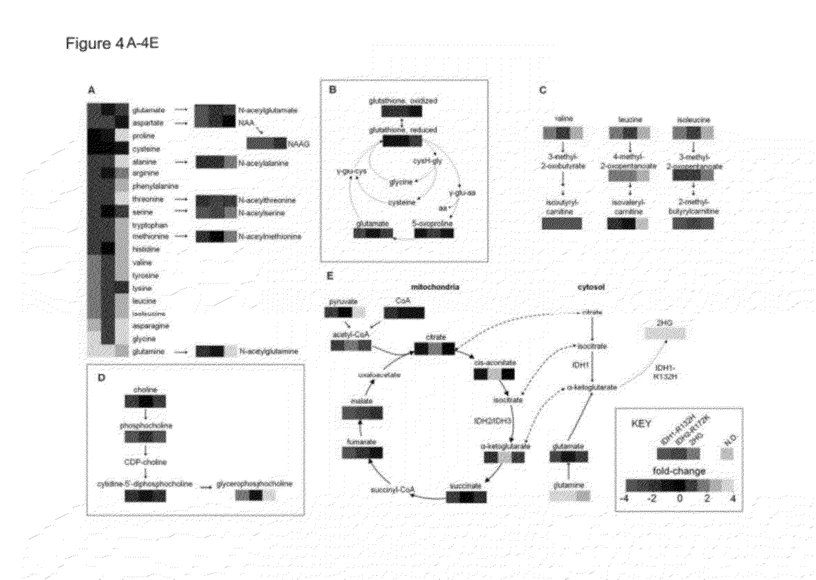

While 2HG treatment and IDH mutant expression induced many similar changes, 56 of the 107 significant alterations that we observed in IDH1-R132H expressing cells were not observed in 2HG-treated cells. 2HG-independent changes included depletion of glutamate and several metabolites that are directly or indirectly derived from glutamate, including glutathiones, N-acetylglutamate, NAAG, α-ketoglutarate, malate, and fumarate. IDH1-R132H expression results in elevated flux from glutamine to 2HG through glutamate and α-ketoglutarate (13) (see pathway in FIG. 4E). Thus, glutamate may become depleted as it is converted first to α-ketoglutarate and then to 2HG. Interestingly, a recent report showed that glioma cells expressing IDH1-R132H are susceptible to knockdown of glutaminase, the enzyme which converts glutamine to glutamate (24). This observation suggests that glutamine to glutamate conversion could be a metabolic bottleneck for IDH-mutated cells. Since treatment with exogenous 2HG does not deplete glutamate, some differences between 2HG-treated and IDH mutant-expressing cells could reflect the different levels of glutamate in these cells. Alternative explanations for the differences between cells expressing IDH mutants and cells treated with 2HG could be that the metabolite profiles reflect different doses of 2HG in these groups, or that IDH mutants exert effects on cellular metabolism that are independent of their enzyme activity.

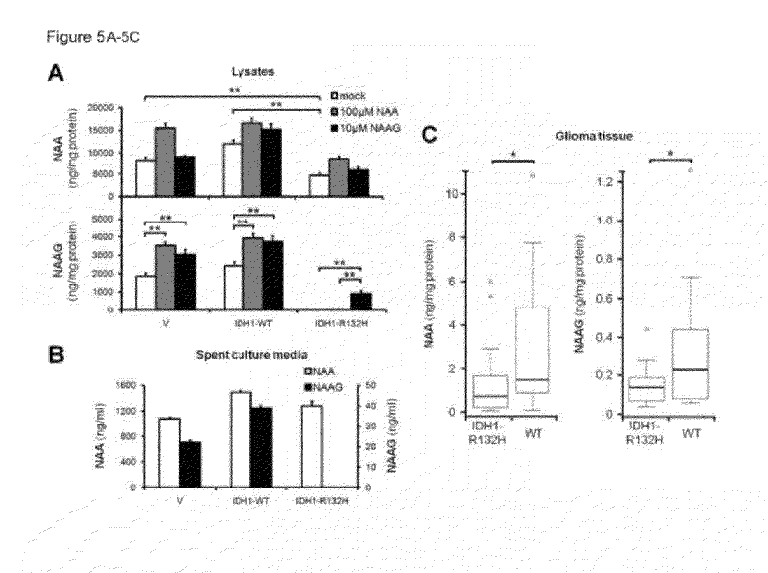

N-acetylated amino acids are lowered in glioma cells expressing IDH1 or IDH2 mutants. Low levels of acetyl-CoA or free amino acids, the substrates for N-acetyltransferases, cannot explain this phenomenon since these compounds were not consistently decreased in cells expressing IDH1-R132H and IDH2-R172K (Tables S1, S4). More likely explanations are that N-acetyltransferase enzymes are downregulated, or breakdown of N-acetylated amino acids is upregulated. NAAG differs from the other N-acetylated amino acids analyzed here in that it is itself synthesized from another N-acetylated amino acid, NAA, by NAAG synthetase. In cells expressing mutant IDH1, NAAG synthetase cannot synthesize NAAG even when NAA is restored to normal levels (FIG. 5A), suggesting that downregulation of this enzyme underlies the depletion of NAAG. Since glutamate is also a substrate for NAAG synthetase, it is also reasonable to expect that this enzyme has a low reaction rate at the low glutamate levels that we observed in cells expressing IDH mutants. Future experiments employing cell-permeable glutamate mimetics could determine whether restoration of normal cellular glutamate levels can rescue NAAG synthetase function in cells expressing IDH mutants. Also, analysis of RNA levels, protein expression, and enzymatic activity for NAAG synthetase and N-acetyltransferases in IDH-mutated gliomas models may further pinpoint the mechanism underlying N-acetylated amino acid depletion.

NAA is the second-most abundant compound in brain, and NAAG is the most abundant dipeptide in brain, but their normal physiological function is poorly understood. Both metabolites take part in a CNS metabolic cycle that includes synthesis of NAAG in neurons, breakdown of NAAG to NAA and glutamate in association with astrocytes, and breakdown of NAA into aspartate and acetate in oligodendrocytes (23, 25). The finding that NAA and NAAG are lowered in a cell line homologously expressing IDH mutants and also in human glioma tissue with IDH1 mutations suggests that glioma cell lines homologously expressing IDH mutants recapitulate features of human gliomas with somatic IDH mutations in situ. The difference in NAA and NAAG levels in IDH1-mutated compared to wild-type tumors (2.4-fold for NAAG) was not as large as the difference we observed for cells expressing IDH1-R132H compared to vector control cells (50-fold for NAAG). However, as cancer cells are “contaminated” with normal vascular and inflammatory cells in glioma tissue (5), normal cells containing higher amounts of NAA and NAAG could mask a more striking difference between the cancer cells themselves. Whether NAA or NAAG depletion contributes to glioma pathogenesis is unclear. However, if NAA or NAAG are found to exert tumor suppressing function that is relieved in IDH-mutated gliomas, therapeutics that replenish these compounds in tumors could have clinical utility.

Some of the most conspicuous features of cells expressing IDH mutants included elevation of numerous free amino acids, elevation of lipid precursors such as glycerol-phosphates and GPC, and depletion of the TCA cycle intermediates citrate, cis-aconitate, a-ketoglutarate, fumarate, and malate. We did not observe changes in intermediates related to glycolysis such as 1,6-glucose phosphate, pyruvate, or lactate that were reproducible and shared between IDH1 and IDH2 mutant expressing cells. Thus, the levels of biosynthetic molecules were increased and TCA intermediates were decreased in cells expressing the either IDH mutant, without consistent accumulation or depletion of glycolytic intermediates. These changes could result from shunting of carbons from glycolysis into de novo synthesis of amino acids and lipids rather than into the TCA. Alternatively, they could reflect a lower rate of amino acid and lipid catalysis into carbon backbones that ultimately enter the TCA. A possible explanation for the increase in free amino acids in cells expressing IDH mutants or treated with 2HG could be that 2HG inhibits α-ketoacid transaminases, which are enzymes that normally transfer amine groups from free amino acids to α-ketoglutarate as a first step in amino acid breakdown for oxidation in the TCA. This possibility is in line with the hypothesis that 2HG can competitively inhibit α-ketoglutarate dependent enzymes based on its structural resemblance to α-ketoglutarate (26). It has been proposed that TCA down-regulation is a major effect of some genetic alterations in cancer, and that this is associated with a selective advantage for cancer cells because nutrients are then converted to building blocks such as amino acids and lipids to be used for proliferation rather than being oxidized in the TCA (4). 2HG treatment of chick neurons has been observed to impair complex V (ATP synthase) of the mitochondrial electron transport chain (27). Thus, a possible mechanism by which IDH mutants dysregulate the TCA could be by producing 2HG that disrupts the normal transfer of electrons from TCA intermediates into the electron transport chain.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Materials And Methods

Stable HOG clones were created by expansion of single cells transduced with lentiviruses or retroviruses for gene or shRNA expression, respectively. Metabolomic profiling was carried out in collaboration with Metabolon. Hierarchical clustering, Welch's t-tests, Pearson correlation, and PCA were performed in R. Human tissue was obtained with consent and analyzed at the Preston Robert Tisch Brain Tumor Center at Duke Biorepository. LC-MS/MS for NAA/NAAG analysis was performed using an Agilent 1200 series HPLC and Sciex/Applied Biosystems API 3200 QTrap in +ESI mode. More details about these are provided below.

Cell lines. HOG cells were derived from a human WHO grade III anaplastic oligodendroglioma (28) and previously found not to contain exon 4 IDH1 or exon 4 IDH2 mutations (8). The HOG cell line was kindly donated by Dr. A. T. Campagnoni at UCLA. To express IDH1 and IDH2 transgenes in cells, IDH1 and IDH2 cDNAs, or cDNAs mutagenized to IDH1-R132H or IDH2-R172K, were cloned into pLenti6.2/V5 (Invitrogen, Carlsbad, Calif.). Viruses were created using these constructs in 293FT cells and these were used to transduce cells derived from the same parental pool of HOG cells for 24 h. Cells derived from the same clone were reasoned to have homogenous IDH transgene expression levels and metabolic profiles compared to pools of cells or transiently transfected cells, facilitating the identification of metabolites that have altered levels in different clones. Virus was replaced by fresh media for 48 hours and then stable clones were selected from single-cell dilutions in 5 μg/ml blasticidin for 3 weeks. Stable HOG cell lines containing IDH1 shRNA or control were constructed by transfecting HOG cells with pSuperRetro vector (OligoEngine, Seattle, Wash.) containing IDH1-specific hairpin or a scrambled sequence (5′-cat aac gag cgg aag aac g-3′). The IDH1-specific hairpin was created using the primers 5′-gat ccc cGG GAA GTT CTG GTG TCA Tat tca aga gaT ATG ACA CCA GAA CTT CCC ttt ttg gaa a-3′ and 5′-agc ttt tcc aaa aaG GGA AGT TCT GGT GTC ATA tct ctt gaa TAT GAC ACC AGA ACT TCC Cgg g-3′, with capital letters representing bases homologous to IDH1 sequence. Clones were selected with 500 μg/ml G418 for 3 weeks and expanded after single cell dilution. Percent knockdown was determined by ImageJ (v1.43, available at http://rsbweb.nih.gov/ij/, developed by Wayne Rasband, National Institutes of Health, Bethesda, Md.) analysis of the intensity of immunoblot anti-IDH1 bands, normalized to the intensity of anti-GAPDH internal control bands (FIG. S4A). The same clone of vector, IDH1-WT, and IDH1-R132H HOG cells were used in all experiments.

2HG synthesis. 2HG was synthesized by treatment of D-glutamate (Sigma-Aldrich, St. Louis, Mo.) with nitrous acid to form a lactone, which was then hydrolyzed with NaOH solution to form 2-D-hydroxyl glutarate. Purity was 93%. Powder was resuspended in PBS and filtered through a 0.22 μm filter using sterile technique for treatment of cells.

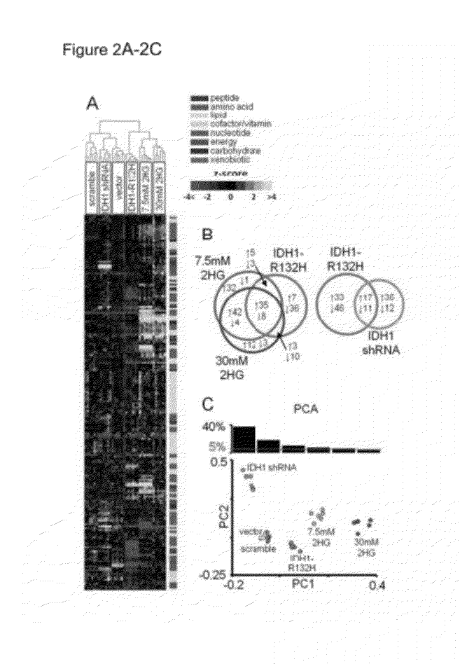

Metabolomic analysis. Cell line treatment: Cells were grown under respective experimental conditions in IMDM media (Gibco, Invitrogen, Carlsbad, Calif.) supplemented with 10% FBS. For the experiment in FIG. 2, cells were grown in a media mix that also contained either 10% PBS or 10% of a 300 mM or 75 mM 2HG solution in PBS for a final 30 mM or 7.5 mM 2HG. Cells were seeded into flasks three days before harvesting. To harvest cells, media was removed, monolayers were washed with PBS, and 0.05% trypsin/EDTA was added. Cells were incubated for 20 min at 37° C. or until cells detached. Two volumes of media were added to the Trypsin/cell mix and suspended by gentle pipetting and triteration. Cells were counted, and 10⁷ cells per sample were spun down at 1000 rpm×3 min in a polystyrene tube. Cells were washed twice with PBS and then snap-frozen on dry ice and stored at −80° C. until analysis.

Metabolite analysis: Metabolomic profiling analysis of all samples was carried out in collaboration with Metabolon (Durham, N.C.) as described previously (29-31), as follows:

Sample Accessioning: Each sample received was accessioned into the Metabolon Laboratory Information Management System (LIMS) and was assigned by the LIMS a unique identifier, which was associated with the original source identifier only. This identifier was used to track all sample handling, tasks, results etc. The samples (and all derived aliquots) were bar-coded and tracked by the LIMS system. All portions of any sample were automatically assigned their own unique identifiers by the LIMS when a new task was created; the relationship of these samples was also tracked. All samples were maintained at −80° C. until processed.

Sample Preparation: The sample preparation process was carried out using the automated MicroLabSTAR® system (Hamilton Company, Reno, Nev.). Recovery standards were added prior to the first step in the extraction process for QC purposes. Sample preparation was conducted using a proprietary series of organic and aqueous extractions to remove the protein fraction while allowing maximum recovery of small molecules. The resulting extract was divided into two fractions; one for analysis by LC and one for analysis by GC. Samples were placed briefly on a TurboVap® (Zymark, Hopkinton, Mass.) to remove the organic solvent. Each sample was then frozen and dried under vacuum. Samples were then prepared for the appropriate instrument, either LC-MS or GC-MS.

QA/QC: For QA/QC purposes, a number of additional samples are included with each day's analysis. Samples included a well-characterized pool of human plasma; a pool of a small aliquot of each experimental sample; an ultra-pure water process blank; and an aliquot of solvents used in extraction to segregate contamination sources in the extraction. Furthermore, a selection of QC compounds is added to every sample, including those under test. These compounds are carefully chosen so as not to interfere with the measurement of the endogenous compounds.

Liquid chromatography/Mass Spectrometry (LC-MS, LC-MS/MS): The LC-MS portion of the platform was based on a ACQUITY HPLC (Waters, Milford, Mass.) and a LTQ mass spectrometer (Thermo-Finnigan, West Palm Beach, Fla.), which consisted of an electrospray ionization (ESI) source and linear ion-trap (LIT) mass analyzer. The sample extract was split into two aliquots, dried, then reconstituted in acidic or basic LC-compatible solvents, each of which contained 11 or more injection standards at fixed concentrations. One aliquot was analyzed using acidic positive ion optimized conditions and the other using basic negative ion optimized conditions in two independent injections using separate dedicated columns. Extracts reconstituted in acidic conditions were gradient eluted using water and methanol both containing 0.1% Formic acid, while the basic extracts, which also used water/methanol, contained 6.5 mM Ammonium Bicarbonate. The MS analysis alternated between MS and data-dependent MS/MS scans using dynamic exclusion.

Gas chromatography/Mass Spectrometry (GC-MS): The samples destined for GC-MS analysis were redried under vacuum desiccation for a minimum of 24 hours prior to being derivatized under dried nitrogen using bistrimethyl-silyl-trifluoroacetamide (BSTFA). The GC column was 5% phenyl and the temperature ramp is from 40° to 300° C. in a 16 minute period. Samples were analyzed on a Thermo-Finnigan Trace DSQ fast-scanning single-quadrupole mass spectrometer using electron impact ionization. The instrument was tuned and calibrated for mass resolution and mass accuracy on a daily basis. The information output from the raw data files was automatically extracted as discussed below.

Accurate Mass Determination and MS/MS fragmentation (LC-MS), (LC-MS/MS): The LC-MS portion of the platform was based on a Waters ACQUITY HPLC and a Thermo-Finnigan LTQ-FT mass spectrometer, which had a linear ion-trap (LIT) front end and a Fourier transform ion cyclotron resonance (FT-ICR) mass spectrometer backend. For ions with counts greater than 2 million, an accurate mass measurement could be performed. Accurate mass measurements could be made on the parent ion as well as fragments. The typical mass error was less than 5 ppm. Ions with less than two million counts require a greater amount of effort to characterize. Fragmentation spectra (MS/MS) were typically generated in data dependent manner, but if necessary, targeted MS/MS could be employed, such as in the case of lower level signals.

Number of biochemicals detected. At the time of publication, the metabolomics platform is capable of detecting approximately 10,000 unique biochemicals, including 2,200 known metabolites, with the remainder consisting of unique metabolites of unknown structure. Only those metabolites present at levels within the range of quantification for a large enough proportion of samples within a batch of samples were analyzed for that batch (see imputing in methods in Statistics section).

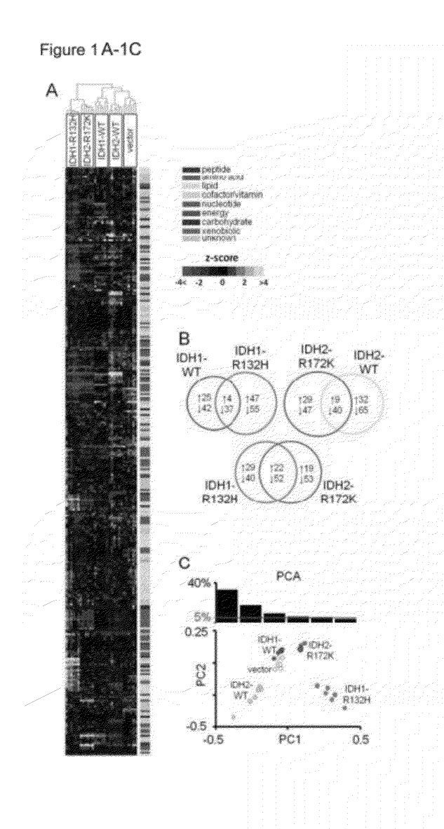

Instrument and process variability. Confounding factors related to technical differences in the behavior of the LC-MS/MS or GC-MS/MS in each sample run (instrumental variability) and to differences in preparation of individual samples (process variability) inevitably result in differences between samples that are unrelated to the biological variable of interest (i.e., 2HG treatment or transgene expression). To provide information on instrument variability, internal standards were added to each sample prior to injection into the mass spectrometers. Then, the median relative standard deviation (RSD) of the ion counts of these standards for all samples in each batch was calculated. Information on overall process variability was provided by calculating the median RSD for all endogenous metabolites (i.e., noninstrument standards) present in 100% of the cell lysate samples in each batch. The median RSD values for the batch of HOG cell lysates expressing IDH1 and IDH2 transgenes (FIG. 1, Table S1) were 6% for internal standards and 12% for endogenous metabolites. Median RSD values for the batch of spent media samples (FIG. S3, Table S3) were 4% for internal standards and 10% for endogenous metabolites. Median RSD values for the batch of HOG cell lysates expressing IDH1-R132H, treated with 2HG, or with IDH1 knockdown (FIG. 2, Table S4) were 6% for internal standards and 14% for endogenous metabolites.

Agreement between sample runs: 179 of the biochemicals detected and analyzed in the first cell lysate metabolomic analysis (Table S1, FIG. 1) were also detected and analyzed in the second cell lysate metabolomic analysis (Table S4, FIG. 2). Some biochemicals were detected in one run and not another due to variation in the lower limit of detection in different runs. Of these 179 biochemicals, 118 were significantly changed (p<0.05) in IDH1-R132H expressing cells in at least one experiment. 100 of these biochemicals were changed in the same direction in both experiments, and 3 biochemicals were significantly changed in opposite directions in either experiment.

Isocitrate dehydrogenase activity assays. Cells were harvested and homogenized in 0.02% Triton-X100 PBS. This was sonicated 3×20 s and protein concentration was quantified. 20 μg cell lysate was added to 1 ml 33 mM Tris-Cl pH 7.5, 2 mM MnCl2, 107 μM NADP⁺ and OD340 nm was measured for 1 min on a UV-2501PC (Shimadzu, Kyoto, Japan). Reactions were performed in triplicate. NADPH production was calculated using NADPH extinction coefficient of 6.2×10³ M⁻¹ cm⁻¹.

Targeted mass LC-MS/MS. Simultaneous quantification of NAA and NAAG in cell culture media, cell lysates, and tissues was done by liquid chromatography-electrospray ionization-tandem mass spectrometry (LC-ESI-MS/MS).

Materials. NAA and NAAG were from Sigma-Aldrich; reagents and solvents were of analytical grade; chromatography solvents of LC-MS grade.

Sample preparation. Media above the cells and cell lysates: To 20 μL of sample 40 μL of ice-cold methanol was added, mixture vigorously agitated (FastPrep, Qbiogene, Carlsbad, Calif. 20 s, speed 6), left at −20° C. for 15 min, agitated again (same cond.), centrifuged at 16,000 g for 5 min, and 50 μL of supernatant dried by vacuum centrifuge (50° C., 1 hr, SpeedVac, Thermo Scientific, West Palm Beach, Fla.). The dry residue was dissolved by 50 μL of mobile phase A (see below) and 10 μL injected into LC-MS/MS system. Tissue samples: To 10-50 μg sample of wet tissue, 300 μL of deionized water and one 4 mm ceramic bead was added in 2-mL polypropylene tube and vigorously agitated (FastPrep, 20 s, speed 4, 2 cycles). A 50 μL aliquot of the homogenate was pipetted out for total protein measurement (for tissue mass normalization purpose) and 500 μL methanol added to the original vial which was again agitated (FastPrep, same conditions), left at −20° C. for 15 min, centrifuged at 16,000 g for 5 min, and 650 μL of supernatant dried by vacuum centrifuge (SpeedVac, 50° C., 1.5 hr). The dry residue was dissolved by 50 μL of mobile phase A (see below) and 10 μL injected into LC-MS/MS system.

LC-MS/MS analysis. Equipment: Agilent 1200 series HPLC (Santa Clara, Calif.) and Sciex/Applied Biosystems API 3200 QTrap (Carlsbad, Calif.). Mobile phase A: water, 3% methanol; mobile phase B: acetonitrile/methanol, 1/1. Analytical column: Kinetex C₁₈, 150×4.6 mm, 2.6 μm, and SafeGuard C₁₈ 4×3 mm guard-column from Phenomenex (Torrance, Calif.). Column temperature: 45° C. Elution gradient at 1 mL/min flow rate: 0-1 min 0% B, 1-2 min 0-80% B, 2-3.5 min 80% B, 3.5-4 min 80-0% B, 4-10 min 0% B. Injection volume: 10 μL. The Q1/Q3 (m/z) transitions monitored in positive electrospray ionization mode: 176/158 (NAA, quantification), 176/134 (NAA, confirmation), 305/148 (NAAG, quantification), 305/130 (NAAG, confirmation).

Calibration. A set of calibrator samples in corresponding matrix was prepared for calibration by adding appropriate amounts of pure NAA and NAAG at the following concentration levels: 0, 0.01, 0.05, 0.25, 1.25, and 6.25 μg/mL. These samples were analyzed alongside the experimental samples and accuracy acceptance criteria was 85% for each but the lowest level (0.01 μg/mL, 80%). The limit of quantification (at 80% accuracy criterion) was determined to be 10 ng/ml for both NAA and NAAG. Samples in which NAA or NAAG was not detected were assigned to have a value of 0 NAA or NAAG (FIG. 5A,B). Statistically significant differences that we reported (FIG. 5A,B) were still significant when 10 ng/ml was assigned for samples in which NAA or NAAG was not detected. Quadratic least squares regression curve fit was employed to account for slight but predictable nonlinearity (ESI of highly polar analytes) with 1/x weighing factor. The addition of methanol to the sample matrix is shared with a recently reported NAAG analysis method (32). Otherwise this procedure is, to the best of our knowledge, novel.

Glioma tissue. Glioma samples were obtained from The Preston Robert Tisch Brain Tumor Center Biorepository at Duke University. Samples were selected based on tissue availability. Samples were analyzed previously for tumor type and IDH mutation status by sequencing exon 4 of IDH1 and exon 4 of IDH2 (8). Samples listed as WT had no mutations in exon 4 of either IDH1 or IDH2. Tissue was carefully dissected by cutting 3-5 mg samples from frozen blocks on dry ice.

Statistics. Impution and normalization. Some biochemicals that were detected in some, but not all, samples in an experiment. Biochemicals that were detected in <50% of replicates in a study group were not analyzed further. For biochemicals that were detected in all samples from one or more groups but not others, the other samples were assumed to have a level of that biochemical near the lower limit of detection. In this case, the lowest detected level of these biochemicals was imputed for samples in which that biochemical was not detected. Quantification values were then normalized to protein concentrations obtained using the Bradford assay. Biochemicals were mapped to pathways based on KEGG, release 41.1, http://www.genome.jp/kegg (20). 2-oleoylglycerol (2-monoolein) was not included in statistical analyses because it was only detected in one of thirty samples in experiment 2.

Univariate statistics. Welch's t-test was used to determine whether mean levels of a biochemical in one experimental group were different from mean levels in another group. The q-value estimates the likelihood that a statistically significant comparison is likely to be a false discovery (33). Q-values are shown in Tables S1, S2, S3 for each comparison. For this study, comparisons with p<0.05, q<0.01 would be estimated to have a false discovery rate estimated to be less 1%. The q-values are listed in the supplemental tables to provide additional information on changes in biochemical abundances, but q-values were not a criterion for the analyses described here. A two-tailed Student's t-test assuming unequal variances was used to determine if a difference existed in the levels of NAA or NAAG between samples (FIG. 5A). NAA and NAAG measurements in lysates and spent media are from four LC-MS/MS readings on two independent experiments, and are representative of six LC-MS/MS readings on three independent experiments (FIGS. 5A, 5B). A one-tailed Student's t-test assuming equal variances was used to determine whether relative mean levels of NAA or NAAG in tumor tissue was significantly lower for tumors with IDH1 mutations than for tumors without IDH1/IDH2 mutations (FIG. 5C). Boxplots (FIG. 56) were created using the boxplot function in R, version 2.12.2 (34).

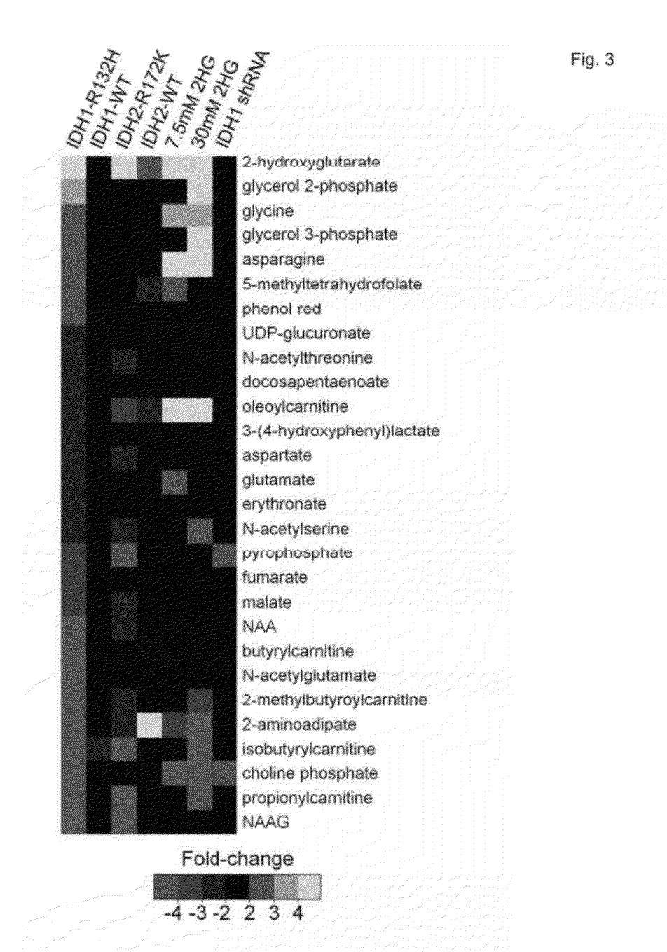

Multivariate statistics. Multivariate statistics and associated graphics were performed in R. Pearson product-moment correlation coefficients (r) were calculated using the cor function (Tables 1, 2). For heat maps (FIGS. 1A, 2A, S2A), Pearson distance was used as the pairwise distance between individual replicates. Pearson distance was calculated as 1−r. Dendrograms were created from this pairwise distance data using the as.dist function, hclust (complete linkage method) function, and as.dendrogram function. Heat maps were drawn using these dendrograms and the heatmap.2 function found in the gplots package. Heat maps of z scores (FIGS. 1A, 2A, S2A) and fold-changes (FIGS. S2D, 3, 4) were plotted using log 2-transformed data. However, the color keys indicate the actual (non-log 2-transformed) values. PCA was performed using the prcomp function (FIGS. 1C, 2C, S1C, S1D, S2C, S4C, S4D). For fold-change of IDH1-R132H cells, the average of this value from two independent experiments is displayed (FIGS. 3, 4).

Removal of outliers. We detected three outlier biochemicals with extremely different values from sample to sample that tended to (1) mask the effects of other biochemicals and (2) exaggerate the similarity that we observed between IDH1 R132H, IDH2-R172K, and 2HG treatment groups. We identified outlier biochemicals as biochemicals that clustered in a separate branch from all other biochemicals in unsupervised hierarchical clustering, had a PCA loading value greater than twice as high as other biochemicals, and were more than 5-fold different in absolute value between at least 2 samples. To better display the complexity of these data, we removed several such outliers based on these criteria. 2HG was removed from all heat maps, PCA, and Pearson correlation calculations (FIGS. 1,2,S1,S2,S3; Tables 1, 2) for this reason. In the dataset derived from spent media samples (FIG. S3), pyrophosphate and methyl-4-hydroxybenzoate also met our criteria for outliers. Also, both metabolites had spurious MS readings between different replicates within several of the sample groups (Table S3). Both of these metabolites were removed from analyses of these data (FIG. S3).

### Example 2

Glioma cells expressing IDH1-R132H and IDH2-R172K have similar metabolomes. To test whether IDH mutants alter the metabolic profile of glioma cells, we performed unbiased metabolic profiling on sister clones of the human oligodendroglioma cell line (HOG) that stably express IDH1-R132H or IDH2-R172K. As controls, we expressed IDH1-WT, IDH2-WT, or vector alone in sister clones (FIG. S1C, S1D). We analyzed lysates prepared from cells in logarithmic growth phase using three mass spectrometry platforms, LC-MS (+/−ESI) and GC-MS (+EI), in six replicates per sample. This yielded MS ion counts corresponding to 315 biochemicals, 215 of which were known metabolites, and 100 of which are unique biochemicals with unknown identity. We normalized these data to protein concentration and mapped the mean level of each biochemical to pathways (Table S1) based on the Kyoto Encyclopedia of Genes and Genomes (KEGG) (20). To determine which clones shared global metabolic profile features, we used unsupervised hierarchical clustering, univariate comparisons, and correlation analysis. We also used PCA, a dimension reduction strategy that transforms a large number of variables, in this case metabolites, into a small number of variables that describe the variation between groups (21). Our procedure for technical and statistical analysis is summarized in FIG. S2.

Hierarchical clustering revealed that IDH1-R132H and IDH2-R172K cells cluster together, separately from controls (FIG. 1A). IDH1-R132H cells and IDH2-R172K cells had 143 and 146 biochemicals with significantly changed (cutoff value for significance: p<0.05, Welch's t test) levels compared to vector control cells, respectively. 74 of these biochemicals were altered in the same direction in both IDH mutant groups, more than for any comparison of an IDH mutant with its respective WT control (FIG. 1B). The levels of biochemicals in these groups had a weak but significant correlation (r=0.15, p=0.008), and did not correlate well with controls (Table S2). PCA showed that IDH1-R132H and IDH2-R172K expressing cells were distinguished from controls by their PC1 value (33.3% of variance, FIG. 1C, S1E, S1F). These analyses demonstrate that IDH1-R132H and IDH2-R172K expression are associated with a specific set of shared metabolic alterations.

We next investigated whether differences in metabolism in cells expressing IDH1-R132H might cause those cells to have altered uptake or excretion of specific metabolites. To test this, we analyzed spent media incubated for 48 hours with HOG clones that express IDH1-R132H, IDH1-WT, or vector, as well as fresh media. Hierarchical clustering, correlation analysis, and PCA of 111 biochemicals in these samples demonstrated that media incubated with cells expressing IDH1-R132H has a distinct metabolic profile from media incubated with controls (FIG. S3A,B,C). In the IDH1-R132H group, 2HG, kynurenine, and glycerophosphocholine (GPC), were increased while the branched-chain amino acid (BCAA) catabolites 4-methyl-2-oxopentanoate, 3-methyl-2-oxovalerate, and 3-methyl-2-oxobutyrate were decreased compared to controls (FIG. S3D). These six metabolites are a subset of those that were altered in lysates of cells expressing either IDH1-R132H or IDH2-R172K (Table S1).

### Example 3

Glioma cells expressing IDH1-R132H share metabolomic features with cells treated with 2HG, but not with cells that have IDH1-WT knockdown. We next sought to provide information on whether any of the known functions of IDH1-R132H could be responsible for the metabolomic changes that we observed. Currently, the two suspected functions of IDH1 mutants are to gain the neomorphic enzymatic activity required to convert α-ketoglutarate to 2HG (13) and to bind IDH1-WT and dominant-negatively inhibit its isocitrate dehydrogenase activity (12). To test whether 2HG alone could produce metabolic changes similar to those resulting from IDH mutant expression, we analyzed cells treated with media containing 7.5 mM or 30 mM 2HG, representing the range of concentrations of 2HG found in IDH1-mutated human glioma tissues (13). To test whether a loss of IDH1-WT function can produce the same metabolite changes as IDH1-R132H expression, we analyzed sister HOG clones that expressed shRNA targeted to IDH1. The IDH1-targeted shRNA reduced IDH1 protein levels by more than 90% and lowered isocitrate dehydrogenase activity accordingly (FIG. S4A). We obtained data on the levels of 204 known biochemicals in cells treated with 2HG and cells stably expressing IDH1-targeted shRNA, as well as analogous control cells (FIG. S4B, Table S4).

Hierarchical clustering revealed that 2HG-treated cells clustered together with IDH1-R132H expressing cells, while IDH1 knockdown and control cells clustered separately (FIG. 2A). The levels of 107, 117, and 130 biochemicals were altered in the IDH1-R132H expression, 7.5 mM 2HG, and 30 mM 2HG groups, respectively, and 43 of these alterations were shared among all three groups (FIG. 2B). Additionally, the biochemical levels were correlated for the IDH1-R132H and 30 mM 2HG group (r=0.22, p=0.001, Table S5). Fewer alterations were shared between the IDH1 knockdown and IDH1-R132H expression groups, and these groups were inversely correlated (r=−0.15, p=0.03). PCA revealed that 2HG treatment and IDH1-R132H expression groups shared large PC1 (37.7% of variance) values compared to the other groups, but that IDH1 knockdown and IDH1-R132H expression did not share any PC loading values that distinguished these groups from controls (FIG. 2C, S4C,D).

To integrate our findings and identify biochemicals that were most altered in cells expressing IDH1-R132H, we analyzed the 28 biochemicals that were reproducibly and significantly altered by 2-fold or more by IDH1-R132H expression (FIG. 3). We found that many of these biochemicals were also altered in cells expressing IDH2-R172K, and to a lesser extent in cells treated with 2HG. However, IDH1-WT, IDH2-WT, and IDH1 shRNA-treated cells shared only a few (range 0 to 2) of these alterations.

### Example 4

Amino acid, choline lipid, and TCA cycle metabolites have altered levels in cells expressing IDH mutants or treated with 2HG. Next, we used information from the above analyses of cell lysates to identify metabolic pathways that were affected by IDH1-R132H expression, IDH2-R172K expression, or 2HG treatment. Because 30 mM 2HG, as opposed to 7.5 mM 2HG, achieved intracellular 2HG levels and global changes more similar to those observed for IDH mutant expression, we chose to focus on this 2HG treatment level. We selected KEGG sub pathways (as delineated in Tables S1 and S4, Heatmap tabs) that had significant and reproducible alterations in >50% of biochemicals from that sub pathway in cells expressing IDH1-R132H. After selecting sub pathways that were altered in cells expressing IDH1-R132H in this manner, we determined the level of metabolites in these sub pathways in the IDH1-R132H, IDH2-R172K, and 2HG-treated cells. We then mapped these data to simplified versions of these pathways (FIG. 4).

This analysis revealed that amino acids and their derivatives were altered in both IDH1-R132H, IDH2-R172K, and 2HG groups (FIG. 4A). Many amino acids, including glycine, serine, threonine, asparagine, phenylalanine, tyrosine, tryptophan, and methionine were increased (range: 1.2- to 5.6-fold, p<0.05) in all three groups. Aspartate, on the other hand, was decreased in all three groups (range: 1.8- to 2.5-fold, p<0.001 for each). Interestingly, glutamate was decreased in the IDH1-R132H (2.6-fold, p<0.001) and IDH2-R172K cells (1.4-fold, p=0.003), but was increased in 2HG-treated cells (1.4-fold, p=0.002). Glutamine was one of only three biochemicals that were significantly altered in opposite directions in two independent analyses of IDH1-R132H cells (p<0.001 for both). We also observed alterations of N-acetylated amino acids, which are amino acid derivatives synthesized by N-acetyltransferases from free L-amino acids and acetyl-CoA, yielding free CoA as a product. All eight N-acetylated amino acids analyzed were lower in IDH1-R132H expressing cells (range: 1.7- to 50-fold, p<0.05 for each), and seven were also lower in IDH2-R172K expressing cells (range: 1.4- to 8.3-fold, p<0.05 for each). In contrast, six N-acetylated amino acids were increased by 2HG treatment (range: 1.2- to 3.0-fold, p<0.05 for each). While NAAG was somewhat lower in 2HG-treated cells (1.8-fold, p<0.001), it was remarkably lower in IDH1-R132H expressing cells (50-fold lower, p<0.001) and IDH2-R172K expressing cells (8.3 fold, p<0.001). N-acetyl-aspartate (NAA) was also greatly reduced in IDH1-R132H expressing cells (3.4-fold, p<0.001) and IDH2-R172K expressing cells (1.4-fold, p<0.001), and was not significantly changed in 2HG-treated cells (1.1-fold lower, p=0.38). Both reduced and oxidized glutathione, an amino acid-derived antioxidant that scavenges reactive oxygen species, were lower in IDH1-R132H and IDH2-R172K expressing cells (>1.6-fold, p<0.001 for all four comparisons), but these compounds were not significantly affected by 2HG treatment (FIG. 4C).

We also noted changes in the BCAAs valine, leucine, and isoleucine, as well as intermediates in their breakdown (FIG. 4B). Leucine, isoleucine and valine were higher in all three groups (range: 1.4- to 3.0-fold, p<0.05 for all nine comparisons). The branched-chain α-keto acids 4-methyl-2-oxopentanoate, 3-methyl-2-oxovalerate, and 3-methyl-2-oxobutyrate can be converted directly from valine, leucine, and isoleucine, respectively, and are intermediates in their degradation. We found that 4-methyl-2-oxopentanoate and 3-methyl-2-oxopentanoate were elevated in IDH2-R172K expressing cells and 2HG-treated cells (range: 1.5- to 2.5-fold, p<0.03 for all four comparisons), and also near-significantly elevated in IDH1-R132H expressing cells (1.7- and 1.4-fold, p<0.10 for both comparisons). At the same time, 4-methyl-2-oxopentanoate, 3-methyl-2-oxovalerate, and 3-methyl-2-oxobutyrate were all >2-fold decreased in culture media incubated with cells expressing IDH1-R132H (p<0.03). While BCAA and branched-chain α-keto acids were elevated in IDH mutant-expressing and 30 mM 2HG-treated cells, further downstream metabolites of BCAA breakdown were lowered in these cells. These metabolites include isobutyrylcarnitine, isovalerylylcarnitine, and 2-methylbutyroylcarnitine (range: 2.5- to 7.7-fold, p<0.005 for all nine comparisons).

IDH mutant expression and 2HG treatment also resulted in alterations of choline lipid synthesis intermediates. In this pathway, choline is converted to choline phosphate, then to CDP-choline, cytidine-5′-diphosphocholine, and then GPC (FIG. 4D), which serves as a precursor for membrane choline phospholipids. Remarkably, choline phosphate was 10-fold lower in IDH1-R132H expressing cells, 1.8-fold lower in IDH2-R172K expressing cells, and 100-fold lower in cells treated with 30 mM 2HG (p<0.001 for each). Conversely, GPC was higher in IDH1-R132H (1.9-fold, p<0.001) and 2HG-treated (3.0-fold, p<0.001) cells, respectively, although it was decreased in IDH2-R172K cells (1.3-fold, p=0.04). GPC was also 1.9-fold elevated in culture media incubated with IDH1-R132H cells (p=0.04, FIG. S3D).

TCA intermediates were markedly affected by IDH mutant expression (FIG. 4E). Most striking were lowered levels of late TCA intermediates fumarate (3.0- and 1.8-fold, p<0.001 and p=0.002) and malate (5.6- and 2.2-fold, p<0.001 for each) in IDH1-R132H and IDH2-R172K cells, respectively. α-ketoglutarate, which is the substrate for production of 2HG by IDH mutants, was non-significantly lower in IDH1-R132H expressing cells (1.8-fold, p=0.11) and non-significantly higher in 30 mM 2HG-treated cells (1.3-fold, p=0.10). As expected, 2HG was highly elevated in all IDH mutant groups, with a 216-fold elevation for IDH1-R132H cells, a 112-fold elevation for IDH2-R172K cells, and a 54-fold elevation in the 30 mM 2HG group (p<0.001 for each).

### Example 5

N-acetylated amino acids are depleted in IDH1-mutated gliomas. One of the most striking findings of our metabolic profiling analysis was the association of lowered N-acetylated amino acids with IDH mutant expression. Using a novel targeted mass LC-MS/MS quantification method, we verified that NAA and NAAG were lower in HOG cells expressing IDH1-R132H (FIG. 5A, mock treatment group). We also noted that NAA and NAAG are normally secreted into culture media by HOG cells, and that HOG cells expressing IDH1-R132H secrete comparable levels of NAA compared to controls, but that cells expressing IDH1-R132H do not secrete detectable levels of NAAG (FIG. 5B). We next sought to provide information on a mechanism that could account for the very low NAAG levels that we observed in cells expressing IDH1-R132H. NAAG is normally synthesized from NAA and glutamate by NAAG synthase (22, 23). HOG cells incubated in media containing 10004 NAA have higher intracellular NAA levels than controls (FIG. 5A, NAA levels in NAA-treated cells), suggesting that NAA can enter the cell from extracellular media. This treatment increased the level of NAA in IDH1-R132H expressing cells to the normal level of NAA in the vector control. However, this restoration of NAA levels did not increase the NAAG in HOG cells expressing IDH1-R132H, indicating that these cells cannot synthesize NAAG from NAA even in the presence of normal NAA levels. Finally, we treated cells with 10 μM NAAG and found that intracellular NAAG levels were increased compared to the no treatment group, as expected (FIG. 5A, see NAAG levels in NAAG-treated cells). Additionally, NAA levels were modestly increased in the NAAG-treated cells in all groups, indicating that IDH1-R132H expression does not interfere with NAAG breakdown into NAA and glutamate (FIG. 5A, NAA levels in NAAG-treated cells). Next, we determined whether NAA or NAAG depletion occurs in IDH1-mutated cells in vivo by analyzing tissue from 26 intermediate-grade gliomas, including 14 astrocytomas and 12 oligodendrogliomas (Table S6). We found that IDH1-mutated tumors had lower mean levels of NAA (2.1-fold, p=0.049) and NAAG (2.4-fold, p=0.019) compared to non-IDH1-mutated tumors (FIG. 5C).

### Example 6

N-acetylated amino acids are depleted in IDH1-mutated gliomas. One of the most striking findings of our metabolic profiling analysis was the association of lowered N-acetylated amino acids with IDH mutant expression. Using a novel targeted mass LC-MS/MS quantification method, we verified that NAA and NAAG were lower in HOG cells expressing IDH1-R132H (FIG. 5A, mock treatment group). We also noted that NAA and NAAG are normally secreted into culture media by HOG cells, and that HOG cells expressing IDH1-R132H secrete comparable levels of NAA compared to controls, but that cells expressing IDH1-R132H do not secrete detectable levels of NAAG (FIG. 5B). We next sought to provide information on a mechanism that could account for the very low NAAG levels that we observed in cells expressing IDH1-R132H. NAAG is normally synthesized from NAA and glutamate by NAAG synthase (22, 23). HOG cells incubated in media containing 100 μM NAA have higher intracellular NAA levels than controls (FIG. 5A, NAA levels in NAA-treated cells), suggesting that NAA can enter the cell from extracellular media. This treatment increased the level of NAA in IDH1-R132H expressing cells to the normal level of NAA in the vector control. However, this restoration of NAA levels did not increase the NAAG in HOG cells expressing IDH1-R132H, indicating that these cells cannot synthesize NAAG from NAA even in the presence of normal NAA levels. Finally, we treated cells with 10 μM NAAG and found that intracellular NAAG levels were increased compared to the no treatment group, as expected (FIG. 5A, see NAAG levels in NAAG-treated cells). Additionally, NAA levels were modestly increased in the NAAG-treated cells in all groups, indicating that IDH1-R132H expression does not interfere with NAAG breakdown into NAA and glutamate (FIG. 5A, NAA levels in NAAG-treated cells). Next, we determined whether NAA or NAAG depletion occurs in IDH1-mutated cells in vivo by analyzing tissue from 26 intermediate-grade gliomas, including 14 astrocytomas and 12 oligodendrogliomas (Table S6). We found that IDH1-mutated tumors had lower mean levels of NAA (2.1-fold, p=0.049) and NAAG (2.4-fold, p=0.019) compared to non-IDH1-mutated tumors (FIG. 5C).

### Example 7

Amino acid, choline lipid, and TCA cycle metabolites have altered levels in cells expressing IDH mutants or treated with 2HG. Next, we used information from the above analyses of cell lysates to identify metabolic pathways that were affected by IDH1-R132H expression, IDH2-R172K expression, or 2HG treatment. Because 30 mM 2HG, as opposed to 7.5 mM 2HG, achieved intracellular 2HG levels and global changes more similar to those observed for IDH mutant expression, we chose to focus on this 2HG treatment level. We selected KEGG sub pathways (as delineated in Tables S1 and S4, Heatmap tabs) that had significant and reproducible alterations in >50% of biochemicals from that sub pathway in cells expressing IDH1-R132H. After selecting sub pathways that were altered in cells expressing IDH1-R132H in this manner, we determined the level of metabolites in these sub pathways in the IDH1-R132H, IDH2-R172K, and 2HG-treated cells. We then mapped these data to simplified versions of these pathways (FIG. 4).

This analysis revealed that amino acids and their derivatives were altered in both IDH1-R132H, IDH2-R172K, and 2HG groups (FIG. 4A). Many amino acids, including glycine, serine, threonine, asparagine, phenylalanine, tyrosine, tryptophan, and methionine were increased (range: 1.2- to 5.6-fold, p<0.05) in all three groups. Aspartate, on the other hand, was decreased in all three groups (range: 1.8- to 2.5-fold, p<0.001 for each). Interestingly, glutamate was decreased in the IDH1-R132H (2.6-fold, p<0.001) and IDH2-R172K cells (1.4-fold, p=0.003), but was increased in 2HG-treated cells (1.4-fold, p=0.002). Glutamine was one of only three biochemicals that were significantly altered in opposite directions in two independent analyses of IDH1-R132H cells (p<0.001 for both). We also observed alterations of N-acetylated amino acids, which are amino acid derivatives synthesized by N-acetyltransferases from free L-amino acids and acetyl-CoA, yielding free CoA as a product. All eight N-acetylated amino acids analyzed were lower in IDH1-R132H expressing cells (range: 1.7- to 50-fold, p<0.05 for each), and seven were also lower in IDH2-R172K expressing cells (range: 1.4- to 8.3-fold, p<0.05 for each). In contrast, six N-acetylated amino acids were increased by 2HG treatment (range: 1.2- to 3.0-fold, p<0.05 for each). While NAAG was somewhat lower in 2HG-treated cells (1.8-fold, p<0.001), it was remarkably lower in IDH1-R132H expressing cells (50-fold lower, p<0.001) and IDH2-R172K expressing cells (8.3 fold, p<0.001). N-acetyl-aspartate (NAA) was also greatly reduced in IDH1-R132H expressing cells (3.4-fold, p<0.001) and IDH2-R172K expressing cells (1.4-fold, p<0.001), and was not significantly changed in 2HG-treated cells (1.1-fold lower, p=0.38). Both reduced and oxidized glutathione, an amino acid-derived antioxidant that scavenges reactive oxygen species, were lower in IDH1-R132H and IDH2-R172K expressing cells (>1.6-fold, p<0.001 for all four comparisons), but these compounds were not significantly affected by 2HG treatment (FIG. 4C).

We also noted changes in the BCAAs valine, leucine, and isoleucine, as well as intermediates in their breakdown (FIG. 4B). Leucine, isoleucine and valine were higher in all three groups (range: 1.4- to 3.0-fold, p<0.05 for all nine comparisons). The branched-chain α-keto acids 4-methyl-2-oxopentanoate, 3-methyl-2-oxovalerate, and 3-methyl-2-oxobutyrate can be converted directly from valine, leucine, and isoleucine, respectively, and are intermediates in their degradation. We found that 4-methyl-2-oxopentanoate and 3-methyl-2-oxopentanoate were elevated in IDH2-R172K expressing cells and 2HG-treated cells (range: 1.5- to 2.5-fold, p<0.03 for all four comparisons), and also near-significantly elevated in IDH1-R132H expressing cells (1.7- and 1.4-fold, p<0.10 for both comparisons). At the same time, 4-methyl-2-oxopentanoate, 3-methyl-2-oxovalerate, and 3-methyl-2-oxobutyrate were all >2-fold decreased in culture media incubated with cells expressing IDH1-R132H (p<0.03). While BCAA and branched-chain α-keto acids were elevated in IDH mutant-expressing and 30 mM 2HG-treated cells, further downstream metabolites of BCAA breakdown were lowered in these cells. These metabolites include isobutyrylcarnitine, isovalerylylcarnitine, and 2-methylbutyroylcarnitine (range: 2.5- to 7.7-fold, p<0.005 for all nine comparisons).

IDH mutant expression and 2HG treatment also resulted in alterations of choline lipid synthesis intermediates. In this pathway, choline is converted to choline phosphate, then to CDP-choline, cytidine-5′-diphosphocholine, and then GPC (FIG. 4D), which serves as a precursor for membrane choline phospholipids. Remarkably, choline phosphate was 10-fold lower in IDH1-R132H expressing cells, 1.8-fold lower in IDH2-R172K expressing cells, and 100-fold lower in cells treated with 30 mM 2HG (p<0.001 for each). Conversely, GPC was higher in IDH1-R132H (1.9-fold, p<0.001) and 2HG-treated (3.0-fold, p<0.001) cells, respectively, although it was decreased in IDH2-R172K cells (1.3-fold, p=0.04). GPC was also 1.9-fold elevated in culture media incubated with IDH1-R132H cells (p=0.04, FIG. S3D).

TCA intermediates were markedly affected by IDH mutant expression (FIG. 4E). Most striking were lowered levels of late TCA intermediates fumarate (3.0- and 1.8-fold, p<0.001 and p=0.002) and malate (5.6- and 2.2-fold, p<0.001 for each) in IDH1-R132H and IDH2-R172K cells, respectively. α-ketoglutarate, which is the substrate for production of 2HG by IDH mutants, was non-significantly lower in IDH1-R132H expressing cells (1.8-fold, p=0.11) and non-significantly higher in 30 mM 2HG-treated cells (1.3-fold, p=0.10). As expected, 2HG was highly elevated in all IDH mutant groups, with a 216-fold elevation for IDH1-R132H cells, a 112-fold elevation for IDH2-R172K cells, and a 54-fold elevation in the 30 mM 2HG group (p<0.001 for each).

### Example 9

Glioma cells expressing IDH1-R132H share metabolomic features with cells treated with 2HG, but not with cells that have IDH1-WT knockdown. We next sought to provide information on whether any of the known functions of IDH1-R132H could be responsible for the metabolomic changes that we observed. Currently, the two suspected functions of IDH1 mutants are to gain the neomorphic enzymatic activity required to convert α-ketoglutarate to 2HG (13) and to bind IDH1-WT and dominant-negatively inhibit its isocitrate dehydrogenase activity (12). To test whether 2HG alone could produce metabolic changes similar to those resulting from IDH mutant expression, we analyzed cells treated with media containing 7.5 mM or 30 mM 2HG, representing the range of concentrations of 2HG found in IDH1-mutated human glioma tissues (13). To test whether a loss of IDH1-WT function can produce the same metabolite changes as IDH1-R132H expression, we analyzed sister HOG clones that expressed shRNA targeted to IDH1. The IDH1-targeted shRNA reduced IDH1 protein levels by more than 90% and lowered isocitrate dehydrogenase activity accordingly (FIG. S4A). We obtained data on the levels of 204 known biochemicals in cells treated with 2HG and cells stably expressing IDH1-targeted shRNA, as well as analogous control cells (FIG. S4B, Table S4).

Hierarchical clustering revealed that 2HG-treated cells clustered together with IDH1-R132H expressing cells, while IDH1 knockdown and control cells clustered separately (FIG. 2A). The levels of 107, 117, and 130 biochemicals were altered in the IDH1-R132H expression, 7.5 mM 2HG, and 30 mM 2HG groups, respectively, and 43 of these alterations were shared among all three groups (FIG. 2B). Additionally, the biochemical levels were correlated for the IDH1-R132H and 30 mM 2HG group (r=0.22, p=0.001, Table S5). Fewer alterations were shared between the IDH1 knockdown and IDH1-R132H expression groups, and these groups were inversely correlated (r=−0.15, p=0.03). PCA revealed that 2HG treatment and IDH1-R132H expression groups shared large PC1 (37.7% of variance) values compared to the other groups, but that IDH1 knockdown and IDH1-R132H expression did not share any PC loading values that distinguished these groups from controls (FIG. 2C, S4C,D).

To integrate our findings and identify biochemicals that were most altered in cells expressing IDH1-R132H, we analyzed the 28 biochemicals that were reproducibly and significantly altered by 2-fold or more by IDH1-R132H expression (FIG. 3). We found that many of these biochemicals were also altered in cells expressing IDH2-R172K, and to a lesser extent in cells treated with 2HG. However, IDH1-WT, IDH2-WT, and IDH1 shRNA-treated cells shared only a few (range 0 to 2) of these alterations.

### REFERENCES

The disclosure of each reference cited is expressly incorporated herein.
1. DeBerardinis, R. J., Mancuso, A., Daikhin, E., Nissim, I., Yudkoff, M., Wehrli, S., & Thompson, C. B. (2007) Proceedings of the National Academy of Sciences of the United States of America 104, 19345-19350.
2. Yun, J., Rago, C., Cheong, I., Pagliarini, R., Angenendt, P., Rajagopalan, H., Schmidt, K., Willson, J. K., Markowitz, S., Zhou, S., et al. (2009) Science 325, 1555-1559.
3. Gao, P., Tchernyshyov, I., Chang, T. C., Lee, Y. S., Kita, K., Ochi, T., Zeller, K. I., De Marzo, A. M., Van Eyk, J. E., Mendell, J. T., et al. (2009) Nature 458, 762-765.
4. Vander Heiden, M. G., Cantley, L. C., & Thompson, C. B. (2009) Science 324, 1029-1033.
5. Louis, D. N., Ohgaki, H., Wiestler, O. D., Cavenee, W. K., Burger, P. C., Jouvet, A., Scheithauer, B. W., & Kleihues, P. (2007) Acta neuropathologica 114, 97-109.
6. Jansen, M., Yip, S., & Louis, D. N. (2010) Lancet neurology 9, 717-726.
7. Parsons, D. W., Jones, S., Zhang, X., Lin, J. C., Leary, R. J., Angenendt, P., Mankoo, P., Carter, H., Siu, I. M., Gallia, G. L., et al. (2008) Science 321, 1807-1812.
8. Yan, H., Parsons, D. W., Jin, G., McLendon, R., Rasheed, B. A., Yuan, W., Kos, I., Batinic-Haberle, I., Jones, S., Riggins, G. J., et al. (2009) The New England journal of medicine 360, 765-773.
9. Reitman, Z. J. & Yan, H. (2010) Journal of the National Cancer Institute 127, 245-246.
10. Ward, P. S., Patel, J., Wise, D. R., Abdel-Wahab, O., Bennett, B. D., Coller, H. A., Cross, J. R., Fantin, V. R., Hedvat, C. V., Perl, A. E., et al. (2010) Cancer cell 17, 225-234.
11. Hartmann, C., Meyer, J., Balss, J., Capper, D., Mueller, W., Christians, A., Felsberg, J., Wolter, M., Mawrin, C., Wick, W., et al. (2009) Acta neuropathologica 118, 469-474.
12. Zhao, S., Lin, Y., Xu, W., Jiang, W., Zha, Z., Wang, P., Yu, W., Li, Z., Gong, L., Peng, Y., et al. (2009) Science 324, 261-265.
13. Dang, L., White, D. W., Gross, S., Bennett, B. D., Bittinger, M. A., Driggers, E. M., Fantin, V. R., Jang, H. G., Jin, S., Keenan, M. C., et al. (2009) Nature 462, 739-744.
14. Gross, S., Cairns, R. A., Minden, M. D., Driggers, E. M., Bittinger, M. A., Jang, H. G., Sasaki, M., Jin, S., Schenkein, D. P., Su, S. M., et al. (2010) The Journal of experimental medicine 207, 339-344.
15. Griffin, J. L., Lehtimaki, K. K., Valonen, P. K., Grohn, O. H., Kettunen, M. I., Yla-Herttuala, S., Pitkanen, A., Nicholson, J. K., & Kauppinen, R. A. (2003) Cancer research 63, 3195-3201.
16. Chen, C., Gonzalez, F. J., & Idle, J. R. (2007) Drug metabolism reviews 39, 581-597.
17. Mazurek, S. (2007) Ernst Schering Foundation symposium proceedings, 99-124.
18. Fan, T. W., Lane, A. N., Higashi, R. M., Farag, M. A., Gao, H., Bousamra, M., & Miller, D. M. (2009) Molecular cancer 8, 41.
19. Lane, A. N., Fan, T. W., & Higashi, R. M. (2008) IUBMB life 60, 124-129.
20. Kanehisa, M. (2009) Genome informatics 23, 212-213.
21. Jolliffee, I. T. (2002) Principal Component Analysis (Springer, New York, N.Y.).
22. Collard, F., Stroobant, V., Lamosa, P., Kapanda, C. N., Lambert, D. M., Muccioli, G. G., Poupaert, J. H., Opperdoes, F., & Van Schaftingen, E. (2010) The Journal of biological chemistry 285, 29826-29833.
23. Becker, I., Lodder, J., Gieselmann, V., & Eckhardt, M. (2010) The Journal of biological chemistry 285, 29156-29164.
24. Seltzer, M. J., Bennett, B. D., Joshi, A. D., Gao, P., Thomas, A. G., Ferraris, D. V., Tsukamoto, T., Rojas, C. J., Slusher, B. S., Rabinowitz, J. D., et al. (2010) Cancer research 70, 8981-8987.
25. Baslow, M. H. (2010) Amino acids 39, 1139-1145.
26. Frezza, C., Tennant, D. A., & Gottlieb, E. (2010) Cancer cell 17, 7-9.
27. Kolker, S., Pawlak, V., Ahlemeyer, B., Okun, J. G., Horster, F., Mayatepek, E., Krieglstein, J., Hoffmann, G. F., & Kohr, G. (2002) The European journal of neuroscience 16, 21-28.
28. Post, G. R. & Dawson, G. (1992) Molecular and chemical neuropathology/sponsored by the International Society for Neurochemistry and the World Federation of Neurology and research groups on neurochemistry and cerebrospinal fluid 16, 303-317.
29. Ryals, J., Lawton, K., Stevens, D., & Milburn, M. (2007) Pharmacogenomics 8, 863-866.
30. Lawton, K. A., Berger, A., Mitchell, M., Milgram, K. E., Evans, A. M., Guo, L., Hanson, R. W., Kalhan, S. C., Ryals, J. A., & Milburn, M. V. (2008) Pharmacogenomics 9, 383-397.
31. Evans, A. M., DeHaven, C. D., Barrett, T., Mitchell, M., & Milgram, E. (2009) Analytical chemistry 81, 6656-6667.
32. Thomas, A. G., Rojas, C. J., Hill, J. R., Shaw, M., & Slusher, B. S. (2010) Analytical biochemistry 404, 94-96.
33. Storey, J. D. & Tibshirani, R. (2003) Proceedings of the National Academy of Sciences of the United States of America 100, 9440-9445.
34. R Core Development Team (2008) in R Foundation for Statistical Computing, Vienna, Austria.

1. A method of characterizing a brain cell sample or blood cell sample of an individual, comprising:
testing the sample for amount of N-acetyl-aspartyl-glutamate (NAAG) or N-acetyl-aspartate (NAA);
comparing the amount of NAAG or NAA in the sample to the amount in corresponding normal cells of the same individual or to similar cells of a control individual that has an IDH1^{+/+}/IDH2^{+/+} genotype;
wherein a sample with a reduced amount of NAAG or NAA indicates that the individual likely has a IDH1 R132 or IDH2 R172 mutation.

2. The method of claim 1 wherein the amount is reduced at least 2-fold.

3. The method of claim 1 wherein the amount is reduced at least 5-fold.

4. The method of claim 1 wherein the amount is reduced at least 10-fold.

5. The method of claim 1 wherein a brain cell sample is tested.

6. The method of claim 1 wherein a blood cell sample is tested.

7. A method of characterizing a brain cell sample, a blood cell sample, a cerebrospinal fluid sample, or blood plasma sample of an individual, comprising:
testing the sample for amount of a metabolite selected from the group consisting of kynurenine, phosphocholine, glycerophosphocholine, 4-methyl-2-oxopentanoate, 3-methyl-2-oxovalerate, and 3-methyl-2-oxobutryate;
comparing the amount of the metabolite in the sample to the amount in a control sample from an individual that has an IDH1^{+/+}/IDH2^{+/+} genotype;
wherein a sample with an increased amount of kynurenine, phosphocholine, or glycerophosphocholine, or a reduced amount of 4-methyl-2-oxopentanoate, 3-methyl-2-oxovalerate, and 3-methyl-2-oxobutryate, indicates that the individual likely has a IDH1 R132 or IDH2 R172 mutation.

8. The method of claim 7 wherein the sample is cerebrospinal fluid.

9. The method of claim 7 wherein the sample is blood plasma.

10. The method of claim 7 wherein the amount of kynurenine and glycerophosphocholine is increased.

11. The method of claim 7 wherein the amount of 4-methyl-2-oxopentanoate, 3-methyl-2-oxovalerate, and 3-methyl-2-oxobutryate is reduced.

12. The method of claim 1 or 7 wherein the likelihood of an IDH1 or IDH2 mutation is used as a prognostic factor.

13. The method of claim 1 or 7 wherein the likelihood of an IDH1 or IDH2 mutation is used as a diagnostic factor.

14. The method of claim 1 or 7 wherein the likelihood of an IDH1 or IDH2 mutation is used as a factor in prescribing an anti-cancer therapy.

15. A method of treating a cancer in an individual, comprising:
administering an agent to the individual, said agent selected from the group consisting of 2-hydroxyglutarate, N-acetyl-aspartate, or N-acetyl-aspartyl-glutamate.

16. The method of claim 15 wherein the agent is delivered systemically.

17. The method of claim 15 wherein the agent is delivered locally to the cancer.

18. The method of claim 15 wherein the agent is delivered by intratumoral injection.

19. The method of claim 15 wherein the cancer is a brain cancer.

20. The method of claim 15 wherein the cancer is a leukemia.

21. The method of claim 15 wherein the cancer has an IDH1 R132 or IDH2 R172 mutation.

22. The method of claim 15 wherein the agent is D-2-hydroxyglutarate.

23. The method of claim 15 wherein the agent is L-2-hydroxyglutarate.

24. The method of claim 15 wherein cancer cells of the individual carry an IDH1 R132 or IDH2 R172 mutation.

25. The method of claim 15 wherein the cancer is selected from the group consisting of glioblastoma, astrocytoma, oligodendrogliomas, and acute myelogenous leukemia.

26. The method of claim 15 wherein the agent is administered in an amount sufficient to reduce the amount of choline phosphate in cancer cells of the individual.

27. The method of claim 26 wherein the choline phosphate is reduced at least 10-fold.

28. The method of claim 26 wherein the choline phosphate is reduced at least 50-fold.

29. The method of claim 26 wherein the choline phosphate is reduced at least 75-fold.

30. The method of claim 15 wherein the agent is administered in an amount sufficient to reduce the amount of oleoylcarnitine, asparagine, glycerol 3-phosphate, or glycerol 2-phosphate in cancer cells of the individual.
